(19) United States
(12) Patent Application Publication
(10) Pub. No.: US 2023/0028688 A1
(43) Pub. Date: Jan. 26, 2023

(54) Electronic Module and Modular System for a Drug Delivery Device

(71) Applicant: Sanofi, Paris (FR)

(72) Inventors: Adam Moyo Harvey-Cook, London (GB); David Aubrey Plumptre, Worcestershire (GB); Oliver Charles Gazeley, Basel (CH); Robert Veasey, Lillington, Leamington Spa (GB)

(21) Appl. No.: 17/782,682

(22) PCT Filed: Dec. 11, 2020
(86) PCT No.: PCT/EP2020/085729

§ 371 Date: Jun. 6, 2022

(30) Foreign Application Priority Data
Dec. 11, 2019 (EP) 19306630.5
Apr. 23, 2020 (EP) 20315209.5

(51) Int. Cl. A61M 5/315; A61M 5/20

(52) U.S. Cl. CPC A61M 5/31548; A61M 5/20; A61M 2205/50

(57) ABSTRACT

An electronic module is configured for attachment to a proximal end of a drug delivery device in a predefined fastening configuration. The drug delivery device comprises an elongated housing extending in a longitudinal direction and comprising a distal end and the proximal end. The electronic module comprises a mechanical coding comprising a mechanical coding feature to engage with a mechanical counter coding feature of a mechanical counter coding provided at the proximal end of the drug delivery device, wherein one of the mechanical coding feature and the mechanical counter coding feature comprises a protrusion extending in the longitudinal direction and wherein the other one of the mechanical coding feature and the mechanical counter coding feature comprises a recess, wherein, in some configurations, the mechanical coding and the mechanical counter coding are operable to prevent a fastening of the electronic module to the drug delivery device.

### CROSS REFERENCE TO RELATED APPLICATIONS

The present application is the national stage entry of International Patent Application No. PCT/EP2020/085729, filed on Dec. 11, 2020, and claims priority to Application Nos. EP 20315209.5, filed on Apr. 23, 2020, and EP 19306630.5, filed on Dec. 11, 2019, the disclosures of which are incorporated herein by reference.

### TECHNICAL FIELD

The disclosure relates to an electronic module configured for attachment to a drug delivery device. The drug delivery device may be an autoinjector or a manually or semi-automatically operated device. The drug delivery device may be an injection device, such as a pen-type injector. The disclosure further relates to a mechanical interface between the electronic module and the drug delivery device. In particular, the disclosure relates to a mechanical coding of the electronic module to engage with a mechanical counter coding of the drug delivery device. In addition, the disclosure further relates to a modular system comprising an electronic module and further comprising a drug delivery device.

### BACKGROUND

Drug delivery devices for setting and dispensing a single or multiple doses of a liquid medicament are as such well-known in the art. Generally, such devices have substantially a similar purpose as that of an ordinary syringe.

Drug delivery devices, such as pen-type injectors, have to meet a number of user-specific requirements. For instance, with patient's suffering chronic diseases, such like diabetes, the patient may be physically infirm and may also have impaired vision. Suitable drug delivery devices especially intended for home medication therefore need to be robust in construction and should be easy to use. Furthermore, manipulation and general handling of the device and its components should be intelligible and easy understandable. Such injection devices should provide setting and subsequent dispensing of a dose of a medicament of variable size. Moreover, a dose setting as well as a dose dispensing procedure must be easy to operate and has to be unambiguous.

For mechanically implemented drug delivery devices but also for electronically implemented drug delivery devices, e.g. injection device equipped with an electric drive, it is desirable to enable a precise, reliable and quasi-automated supervision and/or collection of drug delivery-related data during use of the injection device. Mechanically operated drug delivery and/or injection devices may be equipped with an electronically implemented electronic module serving as an add-on device or data collection device and being configured to monitor user-induced operation of the injection device. Such electronic modules should be rather compact with regards to their geometric size. Generally, such electronic modules can be used as a memory aid and for accurate dose history logging.

Drug delivery devices, such as pen-type injectors may be configured to deliver a variety of drugs at different dose rates (i.e. number of insulin units per click). By providing dedicated electronic modules or add-on devices for data capturing and data logging to particular drug delivery devices, there might be an intrinsic risk of an erroneous pairing of an electronic module with a drug delivery device.

It is therefore an object of the present disclosure to provide an electronic module, a drug delivery device as well as a modular system that comes along with a reduced risk of unsuitably pairing of an electronic module with a drug delivery device. It is therefore an aim to provide an electronic module dedicated for a particular drug delivery device and to ensure, that the electronic module can be only or exclusively mechanically connected or attached to the dedicated drug delivery device; and vice versa.

### SUMMARY

In a first aspect the disclosure relates to an electronic module configured for attachment to a proximal end of a drug delivery device in a predefined fastening configuration. The electronic module is configured for attachment to a drug delivery device comprising an elongated housing extending in a longitudinal direction. The housing comprises a distal end and a proximal end. Typically, the drug delivery device is configured for delivery of a dose of a drug out of the distal end.

The electronic module comprises a mechanical coding. The mechanical coding comprises a mechanical coding feature to engage with a mechanical counter coding feature of a mechanical counter coding provided at the proximal end of the drug delivery device. The mechanical counter coding is provided at the proximal end of the drug delivery device and comprises the mechanical counter coding feature configured to mechanically engage with the mechanical coding feature of the mechanical coding of the electronic module. Mutual engagement of the mechanical coding and the counter mechanical coding is achieved when the mechanical coding matches with the mechanical counter coding and when the electronic module is attached to the proximal end of the drug delivery device in the predefined fastening configuration.

The electronic module may be configured for a detachable fixing and/or for a detachable fastening to the proximal end of the drug delivery device. In that sense, the electronic module may be mechanically coupled or may be coupleable to the drug delivery device in a proximal end region of the drug delivery device.

The technical effect of a proximal attachment of the electronic module to the drug delivery device is that the total elongation or length of the drug delivery device is prolonged only slightly by the attachment of the electronic module whereby an outer diameter of the drug delivery device does not change substantially. When the drug delivery device is implemented as a pen-type injector, a comfortable pen shape is remained that allows for user comfort and easy handling of the drug delivery device.

Generally, the electronic module may be used with several drug delivery devices of equal or of the same type. The assembly or fastening of the electronic module to the drug delivery device is hence only a temporary assembly.

One of the mechanical coding feature and the mechanical counter coding feature comprises a protrusion. The protrusion extends in the longitudinal direction. The other one of the mechanical coding feature and the mechanical counter coding feature comprises a recess. Typically, the protrusion and the recess are complementary shaped such that at least a portion of the protrusion or the entirety of the protrusion is allowed to enter or to penetrate the recess.

When a geometric shape of the protrusion is not matching with a geometric shape of the recess, or when a position of the protrusion in a plane transverse to the longitudinal direction is not matching with a position of the recess in the transverse plane, or even further, when a longitudinal extent of the protrusion is larger than a longitudinal extent of the recess the mechanical coding and the mechanical counter coding are operable to prevent a fastening of the electronic module to the proximal end of the drug delivery device in the predefined fastening configuration.

A mutual fastening of the electronic module and the drug delivery device requires a distally directed movement of the electronic module from a pre-assembly configuration into a final assembly configuration, the latter of which coinciding with the predefined fastening configuration. In case that the mechanical coding of the electronic module does not match with the mechanical counter coding of the drug delivery device at least with regard to any one of the above-mentioned criteria, geometric shape, position in the transverse plane or longitudinal extent, the protrusion is prevented to enter the recess, to engage the recess or to reach a final assembly position inside the recess, thereby blocking and/or impeding a final distally directed movement of the electronic module relative to the drug delivery device from the pre-assembly configuration towards and into the final assembly configuration or predefined fastening configuration.

For successfully pairing and for fastening of the electronic module to the drug delivery device it is required that the mechanical coding feature matches the counter mechanical coding feature. A mutual assembly and hence a distally directed displacement of the electronic module relative to the drug delivery device from the pre-assembly configuration, in which the mechanical coding is longitudinally aligned with the mechanical counter coding towards and into the predefined fastening configuration is only possible when the geometric shape of the protrusion matches with the geometric shape of the recess and/or when the lateral or transverse position of the protrusion matches the lateral or transverse position of the recess. The transverse or lateral direction extends perpendicular to the elongation of the housing of the drug delivery device.

With some examples, the mechanical coding feature is a keying feature to engage with a correspondingly shaped keying feature of the mechanical counter coding. In configurations, wherein the mechanical coding does not match with the mechanical counter coding, the mechanical coding feature and/or the mechanical counter coding feature serve and behaves as a blocking feature configured to prevent mutual attachment of the drug delivery device and the electronic module.

Generally, there may be provided a kit of numerous electronic modules, that distinguish by their mechanical coding. A first mechanical coding of a first electronic module distinguishes from a second mechanical coding of a second electronic module. The first mechanical coding comprises a first mechanical coding feature. The second mechanical coding comprises a second mechanical coding feature. Accordingly, the first mechanical coding feature comprises at least one of a first protrusion and a first recess. The second mechanical coding feature comprises at least one of a second protrusion and a second recess. The geometric shape of the first protrusion may distinguish from the geometric shape of the second protrusion. Additionally or alternatively, a position of the first protrusion in a plane transverse to the longitudinal direction distinguishes from a position of the second protrusion in the transverse plane. The same applies to the first and second recesses of first and second electronic modules.

Accordingly, the present disclosure also relates to a set of drug delivery devices that distinguish by their mechanical counter coding. There may be provided at least a first drug delivery device with a first mechanical counter coding and a second drug delivery device with a second mechanical counter coding. The first mechanical counter coding matches with the first mechanical coding but does not match with the second mechanical coding. Likewise, the second mechanical counter coding only matches with the second mechanical coding but does not match with the first mechanical coding. The first and the second mechanical counter coding of first and second drug delivery devices distinguish by at least one of a geometric shape and a transverse position of the respective counter coding features, hence by their shape and/or transverse position of their protrusion or recess.

In this way, it can be guaranteed, that a dedicated electronic module provided with a particular mechanical coding can be only mechanically engaged or mechanically paired with a drug delivery device that is provided with a matching mechanical counter coding. The mechanical coding of the electronic module and the complementary-shaped mechanical counter coding of the drug delivery device may prevent the assembly of an electronic module to a drug delivery device or injection device other than the intended or dedicated drug delivery device or injection device.

Here, a rather robust and failure safe modular system can be provided that prevents mismatches between an electronic module and a drug delivery device that does not require or rely on a complex or costly electronically implemented pairing check. With existing modular systems comprising an electronic module and a drug delivery device it may be only required to modify a limited number of plastic injection molded components of the electronic module and/or of the drug delivery device in order to implement the mechanical coding and the mechanical counter coding, respectively. Implementation of the coding and the counter coding may be provided at relatively low cost but with high strength and reliability.

According to a further example, the electronic module comprises a fastening element configured to mechanically engage with a complementary-shaped counter fastening element of the drug delivery device in the predefined fastening configuration. The fastening element and the counter fastening element may define the predefined fastening configuration, in which the electronic module is attachable, coupleable or connectable to the drug delivery device. A position and/or a geometric shape of the fastening element of the electronic module typically matches with the geometric shape and/or with the position of the counter fastening element of the drug delivery device. A mutual assembly, hence arranging the electronic module on the drug delivery device in the predefined fastening configuration requires that the fastening element mechanically engages with the complementary-shaped counter fastening element. When the fastening element and the complementary-shaped counter fastening element are in mechanical engagement, the electronic module is in a predefined orientation as well as in a predefined position relative to the drug delivery device.

With some examples, the mechanical coding may be part of the fastening element and the respective counter coding may be part of the counter fastening element. With some examples, the fastening element may provide or may be the mechanical coding and the counter fastening element may provide or may be the mechanical counter coding.

With some examples, the fastening element is separate from the mechanical coding and the counter fastening element is separate from the mechanical counter coding. With some examples, and when a comparatively large variety of electronic modules and drug delivery devices is provided, a set of electronic modules comprises numerous electronic modules each of which comprising a different mechanical coding but each of which comprising identical fastening elements. The same applies to a variety of available drug delivery devices and hence to a set of drug delivery devices. With a set of drug delivery device the individual drug delivery devices may distinguish with regard to their mechanical counter coding but may comprise an identical counter fastening element complementary shaped to the respective fastening element of the electronic module.

With some examples, there is provided a set of electronic modules comprising a first electronic module and a second electronic module. The first electronic module comprises a first mechanical coding. The second electronic module comprises a second mechanical coding. First and second mechanical coding distinguish, e.g. with regards to their geometric shape and/or with regards to the transverse position of at least one of a protrusion and a recess of their respective mechanical coding feature.

First and second electronic modules comprise an identical fastening element. Hence, the fastening element of the first electronic module and the fastening element of the second electronic module are substantially identical, both in terms of their geometric shape as well as in terms of their transverse position with regard to the shape or housing of the electronic module.

The same may apply for a set of drug delivery devices with a first drug delivery device and a second drug delivery device, wherein the first drug delivery device comprises a first mechanical counter coding and wherein the second drug delivery device comprises a second mechanical counter coding. The first and the second mechanical counter codings distinguish, e.g. in terms of their geometric shape and/or with regards to a transverse position of at least one of a protrusion and a recess of the respective first and second mechanical counter coding features. The first and the second drug delivery device each comprise a counter fastening element. The counter fastening elements of the first and the second drug delivery devices may be substantially identical. They may be of identical geometric shape and they may be located at identical positions in the transverse plane.

Providing of an electronic module with a fastening element configured to engage with a complementary-shaped counter fastening element is effective to obtain a well-defined fastening configuration for a variety of electronic modules to be mechanically connected with a variety of drug delivery devices in general. By spatially separating the mechanical coding from the fastening element, the fastening element does not have to be subject to a geometric modification for providing the desired mechanical coding. In this way, an existing fastening mechanism for fastening the electronic module to the drug delivery device can remain unamended. By providing the mechanical coding in a non-overlapping configuration with the fastening element, a rather cost efficient and straightforward approach is provided to mechanically encode the electronic module.

The fastening element may comprise a clip feature and may form a clip connection with the correspondingly or complementary-shaped counter fastening element. Hence, the counter fastening element of the drug delivery device may also comprise a clip feature and may contribute to a click connection between the electronic module and the drug delivery device. With other examples, the fastening element may be configured to establish a friction fit or a force fit with the complementary-shaped counter fastening element of the drug delivery device.

According to another example of the electronic module the mechanical coding is defined by at least one of a position, an orientation and a longitudinal extent of the mechanical coding feature relative to the fastening element. With some examples, the fastening element provides a symmetry breaking feature of the electronic module. The electronic module may be of tubular or disc-like shape. It may comprise a circular shaped cross-section and may be hence rotationally symmetric with regards to the longitudinal direction as an axis of symmetry or as an axis of rotation.

When there is provided a set of numerous electronic modules, e.g. a first electronic module and a second electronic module, respective first and second mechanical codings distinguish by at least one of the position, the orientation and the longitudinal extent of respective first and second mechanical coding features relative to the fastening element. In other words, the mechanical coding feature of a first mechanical coding of a first electronic module distinguishes with regard to at least one of its position, orientation and longitudinal extent relative to the fastening element compared to a respective position and/or orientation of the second mechanical coding feature of a second mechanical coding relative to the fastening element.

The same or similar applies to the mechanical counter coding of a respective drug delivery device. Also, the mechanical counter coding is defined by at least one of a position, an orientation and a longitudinal extent of its mechanical counter coding feature relative to the counter fastening element of the drug delivery device. By defining the mechanical coding relative to the mechanical coding feature, each electronic module of a set of electronic modules can be characterized without a necessity to compare the electronic modules among each other. Generally, a mechanical code may be defined by e.g. a distance of the mechanical coding feature to the fastening element of the electronic module. The distance may be at least one of a radial distance, a circumferential distance and a longitudinal distance between the mechanical coding feature and the fastening element. Typically, there is provided a set of electronic modules comprising a first and a second electronic module wherein at least one of an axial distance, a radial distance and a circumferential distance of a first mechanical coding feature of the first electronic module relative to the fastening element of the first electronic module distinguishes and differs from at least one of a radial distance, a circumferential distance and an axial distance between a second mechanical coding feature relative to the respective fastening element of the second electronic module.

In this way, arranging the electronic module in the predefined fastening configuration and attaching the electronic module to the drug delivery device is only possible when the mechanical coding matches with the mechanical counter coding.

According to a further example the electronic module comprises at least one longitudinal extension extending distally from a distal end of the electronic module. The at least one longitudinal extension is configured to extend into or through an aperture at or near the proximal end of the drug delivery device. The extension may be an extension for a sensor located in or on the electronic module. The extension may be for instance a light guide that is attached to a circuit board. With other examples, the extension may be implemented as a switch, e.g. for power management of the electronic module. When in the predefined fastening configuration, the longitudinal extension of the electronic module may extend distally beyond or across the proximal end of the drug delivery device. In the predefined fastening configuration the extension may be interdigitated with the proximal end or proximal end section of the drug delivery device. The extension may allow use of a drug delivery device that has a movable and/or rotatable part that is used for the detection of selected or delivered doses of the drug.

The longitudinal extension reaching into the proximal end of the drug delivery device may be configured to cooperate with an encoder provided on the movable and/or rotatable part of the drug delivery device.

According to a further example the mechanical coding is defined by at least one of a position, an orientation and a longitudinal extent of the mechanical coding feature relative to the longitudinal extension. Here, the longitudinal extension may provide a symmetry breaking feature of the electronic module. Additionally or alternatively it may be the longitudinal extension that may define the predefined fastening configuration of the electronic module and the drug delivery device when the longitudinal extension is engaged with, extends into or through the aperture at the proximal end of the drug delivery device. The predefined fastening configuration in which the electronic module is attachable or connectable to the drug delivery device may be either defined by the position of the fastening element and the counter fastening element or it may be alternatively defined by the longitudinal extension and the complementary-shaped aperture of the drug delivery device.

With some examples, the electronic module comprises both, a fastening element complementary shaped to the counter fastening element and the longitudinal extension to extend into or through the aperture of the drug delivery device.

According to a further example the electronic module does not comprise only one but numerous fastening elements configured to mechanically engage with a respective number of complementary shaped and complementary arranged counter fastening elements of the drug delivery device in the predefined fastening configuration. With some examples, the electronic module comprises two fastening elements arranged at geometrically opposite position on or near an outside circumference of the housing of the electronic module.

With some examples the electronic module comprises three, four or even more fastening elements, each of which complementary shaped to a respective number of counter fastening elements of the drug delivery device. When there are provided numerous fastening elements the fastening elements may be equidistantly spaced along a circumference of the housing of the electronic module. In this way, a rather robust and failure safe mechanical attachment of the electronic module to the drug delivery device can be provided.

According to a further example the mechanical coding feature comprises the protrusion protruding in longitudinal distal direction from a distally facing surface of the electronic module. Providing the protrusion on the electronic module is beneficial in that the mechanical counter coding feature of the matching drug delivery device is provided with a complementary-shaped recess at the proximal end of the drug delivery device. Hence, the drug delivery device is void of longitudinally extending protrusions at its proximal end, which may be beneficial for a use of the drug delivery device when the electronic module is not provided or is not attached to its proximal end. The protrusion of the mechanical coding feature is typically configured to engage with a complementary-shaped recess of the mechanical counter coding feature. The mechanical counter coding feature and hence the recess is typically provided in or adjacent to a proximally facing surface of the drug delivery device

According and in a further example the distally facing surface of the electronic module is in longitudinal or axial abutment with a complementary shaped proximally facing surface of the drug delivery device when in the predefined fastening configuration. With some examples, the protrusion of the electronic module and the recess of the drug delivery device are shaped such that upon reaching of the predefined fastening configuration the distally facing surface of the electronic module abuts with the proximally facing surface of the drug delivery device. In this way, a rather stable and robust mutual abutment configuration between the electronic module and the drug delivery device can be reached. Mutual abutment of the distally facing surface with the proximally facing surface provides a tilt-free mechanical attachment of the electronic module to the proximal end of the drug delivery device.

With some examples, the mechanical counter coding feature, e.g. the respective recess is provided on an inside surface of a sidewall of the drug delivery device or on an outside surface of the sidewall of the drug delivery device. The recess may comprise a longitudinal slot having a radial depth that matches with the geometric shape of the protrusion of the mechanical coding feature of the electronic module.

In the predefined fastening configuration, the protrusion of the mechanical coding feature may comprise a radially inwardly facing engaging section configured to engage with a recess or slot provided at an outside surface of the sidewall of the drug delivery device. Likewise, the longitudinal protrusion of the mechanical coding feature may comprise a radially outwardly facing engaging section or engaging portion configured to engage with a recess or slot provided on the inside facing inside surface of the sidewall of the drug delivery device. Respective slots provided on the inside or outside surfaces of a sidewall of the drug delivery device may extend towards a proximal end of the respective sidewall. At a proximal end, the sidewall may comprise a radially outwardly or radially inwardly extending flange acting as a longitudinal end stop of the respective recess or slot.

Hence, when the slot is formed as a longitudinal groove on the outside surface of the sidewall of the drug delivery device it may terminate in a radially outwardly extending flange at the proximal end of the drug delivery device. Here, the flange or flange portion terminating the slot or recess in longitudinal proximal direction may form or constitute a snap or clip feature to engage with a correspondingly-shaped snap feature of the longitudinal protrusion of the coding feature in the predefined fastening configuration.

In this way, the coding feature and the correspondingly shaped counter coding feature may provide or may support a fixing and/or a mechanical attachment of the electronic module to the drug delivery device with respect to the longitudinal direction.

According to another example the mechanical coding of the electronic module comprises a coding section. The coding section comprises a number of n discrete spatially non-overlapping coding feature positions and a number of k mechanical coding features. Each one of the coding features is located on one of the coding feature positions. The size of the coding features matches with the size of the coding feature positions. The coding feature positions may be located adjacently next to each other, e.g. they may adjoin in a circumferential or radial direction or along combinations thereof.

The coding feature positions may be arranged along a line or a curve. Hence, a first coding feature position may be located next to a second coding feature position. The second coding feature position may be arranged next to a third coding feature position. The second coding feature position may be arranged between the first and the third coding feature position. The third coding feature position may be arranged adjacent or adjoining to a fourth coding feature position. The third coding feature position may be arranged between the second and the fourth coding feature position.

Typically, n and k being integer numbers. Moreover n≤k or n<k. With some examples n=k/2.

Generally, if the total number of discrete spatially non-overlapping coding feature positions is n, there are 2ⁿ possible configurations and arrangements. But not all of these arrangements may guarantee a unique and dedicated pairing between the electronic device and the drug delivery device. The condition to ensure that that all but one of the electronic modules 620 is hindered to engage with a drug delivery device is that every mechanical coding 650 comprises the same number of mechanical coding features 651, 653. The number of unique permutations of k coding features in n coding feature positions is equal to the binomial coefficient according to the following equation:

$$( \begin{matrix}n \\ k\end{matrix} ) = \frac{n !}{k ! ( n - k ) !}$$

The maximum number of available combinations is obtained when k=n/2.

The coding section with a number of mechanical coding features that may be arranged on one of numerous available coding feature positions provides a kind of binary code. When there is no coding feature at a coding feature position this represents a digital 0. When there is provided a coding feature at a coding feature position this represents a digital 1. Generally, with two coding feature positions in the coding section there may be provided a maximum of four different mechanical codings. With three coding feature positions there may be provided up to 8 different mechanical codings and with four coding feature positions in or on a coding section there may be provided up to 16 different mechanical codings. Generally, the number of available mechanical codings is given by n².

Of course, the mechanical counter coding comprises an inverse arrangement of mechanical counter coding features on or in a mechanical counter coding section.

According to a further example the mechanical coding features of a coding section of a mechanical coding of the electronic module are configured to engage with a respective mechanical counter coding of the drug delivery device, wherein the mechanical counter coding comprises a counter coding section. The counter coding section comprises a number of M discrete spatially non-overlapping counter coding feature positions and a number of L mechanical counter coding features. Each one of the mechanical counter coding features is located on one of the counter coding feature positions. Here, M and L being integer numbers, wherein L≤M or wherein L<M. With some examples, L=M/2.

According to another example of the electronic module the number n of coding feature positions of the coding section equals the number of M counter coding feature positions of the mechanical counter coding and/or the number k of mechanical coding features provided in the coding section equals the number L of mechanical counter coding features provided on or in the mechanical counter coding section.

In another aspect the disclosure relates to a drug delivery device. The drug delivery device comprises a housing. The housing comprises a distal end and a proximal end. The proximal end is configured for attachment of an electronic module as described above in a predefined fastening configuration. The drug delivery device further comprises a drive mechanism. The drive mechanism is configured to set and/or to deliver a dose of a drug out of the distal end. Typically, the proximal end of the drug delivery device is provided at one longitudinal end of the housing and the distal end of the drug delivery device is provided at an oppositely located longitudinal end of the housing.

The drug delivery device comprises a mechanical counter coding provided at the proximal end. The mechanical counter coding comprises a mechanical counter coding feature to engage with the mechanical coding feature of the above-described mechanical coding of the above-described electronic module when attached to the proximal end of the drug delivery device in the predefined fastening configuration.

Generally, the drug delivery device is configured and designed with regard to its counter coding for engaging with the electronic module as described above. Insofar any features, benefits and effects as described above in connection with the electronic module equally apply to the drug delivery device; and vice versa.

The counter coding feature may comprise a protrusion extending in the longitudinal direction or comprises a recess to engage and to receive a respective protrusion of the coding feature. The protrusion or the respective recess is complementary-shaped or is in a complementary position to the respective recess or protrusion of the coding feature of the electronic module. When the mechanical counter coding feature comprises a longitudinally extending protrusion the coding feature of the electronic module comprises a recess. When the mechanical counter coding comprises a recess, the mechanical coding comprises a complementary shaped longitudinal protrusion.

With a matching pair of a mechanical coding and a mechanical counter coding the geometric shape of the protrusion is matching with the geometric shape of the recess and the position of the protrusion in a plane transverse to the longitudinal direction is matching with a respective position of the recess in the transverse plane. Otherwise and when at least one of the geometric shape of the protrusion and recess or at least one of the transverse position of the protrusion and the recess do not mutually match or align when the electronic module is in a pre-assembly configuration, in which the fastening element and the counter fastening element of the electronic module and the drug delivery device are at least longitudinally aligned, a mechanical pairing of the electronic module and the drug delivery device is effectively impeded and blocked. Then, the protrusion may abut with a border region of the recess and/or the geometric shape of the protrusions is not allowed to enter the recess in longitudinal direction.

According to a further example the drug delivery device comprises a counter fastening element configured to mechanically engage with the complementary-shaped fastening element of the electronic module in the predefined fastening configuration. Also here, the counter fastening element may define the predefined fastening configuration. The fastening configuration of the electronic module and the drug delivery device may be defined when the fastening element engages with the complementary-shaped counter fastening element.

With some examples, the drug delivery device and the electronic module each comprise numerous fastening elements, e.g. two fastening elements arranged along the circumference of a housing of the electronic module and along a circumference of the housing of the drug delivery device, respectively. When there are provided two or even more fastening elements they may be located at diametrically opposite positions on the electronic module and/or on the drug delivery device. In this way, there may be provided not only a single and unique predefined fastening configuration but there may be provided e.g. two or more predefined fastening configurations.

The predefined fastening configuration may be characterized by an orientation of the electronic module relative to the drug delivery device, in particular relative to the proximal end of the drug delivery device, with regards to an axis of rotation coinciding with or extending parallel to the longitudinal direction of the housing of the electronic module and/or of the housing of the drug delivery device.

With two fastening configurations it is of particular benefit, with the electronic module comprises two mechanical codings and when the drug delivery device comprises two complementary-shaped mechanical counter codings. It may be even conceivable, that the electronic module comprises three or even four fastening elements equidistantly arranged along the circumference of the housing of the electronic module. Correspondingly, the drug delivery device may then comprise respective three or four counter fastening elements at its proximal end.

With an increasing number of fastening elements there may be also provided a respective increasing number of mechanical codings and/or mechanical counter codings on the electronic module and on the drug delivery device, respectively.

If the fastening elements and the counter fastening elements are equidistantly arranged along the circumference of the electronic module and of the drug delivery device, respectively, there may be provided numerous predefined fastening configurations.

With a number of C predefined fastening configurations there may be provided a respective integer number of mechanical codings on the electronic module. In order to prevent an unsuitable pairing of an electronic module with a drug delivery device it may be sufficient, when the matching drug delivery device comprises only one or more mechanical counter codings to engage with only one of the mechanical codings of the electronic module.

With other examples, it may be the drug delivery device that comprises a number of C mechanical counter codings. Then, it may be sufficient when the electronic module comprises one or more mechanical codings to engage with any one of the mechanical counter codings of the drug delivery device.

According to another example the mechanical counter coding is defined by at least one of a position, an orientation and a longitudinal extent of the mechanical counter coding feature relative to the counter fastening element.

According to a further example, the drug delivery device comprises an aperture to receive at least one longitudinal extension of the electronic module. When in the predefined fastening configuration the aperture at the proximal end of the drug delivery device is configured to receive or to align with the longitudinal extension of the electronic module. Here, and according to a further example, the mechanical counter coding may be defined by at least one of a position, an orientation and a longitudinal extent of the mechanical counter coding feature relative to the aperture at the proximal end of the drug delivery device.

At least one of the counter fastening element and the aperture at the proximal end of the drug delivery device may define at least one or several predefined fastening configurations of the electronic module and the drug delivery device. Accordingly, at least one of the counter fastening element and the aperture at the proximal end of the drug delivery device may serve as a reference for the definition of different counter codings for the drug delivery device. Different counter codings distinguish from each other by varying positions, orientations or longitudinal extents of respective counter coding features relative to at least one of the counter fastening element and the aperture at the proximal end of the drug delivery device.

According to another example the mechanical counter coding feature comprises the recess configured to receive or to engage with the complementary shaped and distally extending protrusion of the coding feature of the mechanical coding of the electronic module when in the predefined fastening configurations. In this way, the proximal end of the drug delivery device may be void of any protrusions. It may be hence configured to receive a longitudinally, typically a distally extending protrusion of the electronic module serving as a mechanical coding feature. Avoiding of the implementation of a longitudinally extending protrusion at the proximal end of the drug delivery device is somewhat beneficial for the general handling of the device and the associated user comfort, in particular when the drug delivery device should be used without the electronic module.

In another example the recess providing the mechanical counter coding feature is located and provided in at least one of a proximally facing surface of the drug delivery device, an outside surface of a sidewall of the drug delivery device and an inside surface of the sidewall of the drug delivery device. When the recess is located at a sidewall of the drug delivery device, e.g. on a sidewall of a dose dial or on the sidewall of an actuating element movably arranged on the proximal end of the drug delivery device the recess may be implemented as a longitudinal slot or groove to receive and/or to engage with the longitudinally extending coding feature of the mechanical coding of the electronic module.

A recess on an inside surface or outside surface of an e.g. tubular or sleeve-shaped component of the drug delivery device may be rather easy to be realized and implemented in an injection molded plastic component. The recess may be provided as a longitudinal groove or slot in or on an inside surface or outside surface of a component of the drug delivery device. The same or similar features may be provided on the electronic module, namely when the mechanical coding feature of the electronic module comprises a respective recess and wherein the mechanical counter coding feature of the drug delivery device comprises a longitudinally extending protrusion.

Generally, there are many different configurations for the protrusion and for the recess. With some examples, the protrusion comprises a rather straight shaped elongated pin. The protrusion may comprise a rectangular shaped slab or tap. The protrusion may comprise a particular geometric structure. Hence, the protrusion may comprise a tubular-shaped sleeve-like, e.g. a circular or oval shaped hollow structure. With other examples, the protrusion comprises a rectangular or triangular cross-section. With further examples, the protrusion may comprise a crossed-structure in the transverse plane formed by two slab-like or planar shaped elements crossing each other.

Correspondingly, the recess may comprise one of a blind hole in a distally or proximally facing surface of one of the electronic module at the drug delivery device. With other examples, the recess comprises a longitudinally extending groove or slot extending along an inside or outside facing sidewall of the housing or of an actuating or adjusting element. Typically, the mechanical coding feature is or forms a keying feature that is complementary or correspondingly shaped to a respective counter keying feature provided by the mechanical counter coding feature.

According to another example the drug delivery device comprises a drug container filled with the drug. The drug may be provided in liquid form inside of the drug container. The drug container may comprise at least one of a syringe, a carpule or a cartridge. The drug container typically comprises a barrel sealed in distal direction and sealed in proximal direction. Towards the proximal direction the drug container or barrel may be sealed by a movable stopper. The movable stopper may be movable relative to the sidewall of the barrel by a piston rod and a driving mechanism of the drug delivery device. The distal end of the barrel or of the drug container may be permanently or temporarily connected to a dispenser, such as an injection needle or an infusion line. With some examples and when the drug delivery device is implemented as a pen-type injection device, the distal end of the housing of the drug delivery device is configured to receive a double-tipped injection needle configured to pierce or to penetrate a distal seal of the drug container when mounted to the housing of the drug delivery device.

The drug delivery device may be implemented as a reusable device, wherein the drug container is configured to be exchanged. With other examples the drug delivery device is implemented as a disposable device. Here, the drug container is not intended to be exchanged. Rather, the entire drug delivery device is intended to become discarded when the content of the drug container has been used. Before discarding of the drug delivery device the electronic module may be detached from the proximal end of the drug delivery device and may be connected or attached to another drug delivery device. With disposable drug delivery devices, the drug container filled with the drug may be readily assembled inside the drug delivery device upon delivery to end consumers or patient against.

According to another aspect the disclosure relates to a modular system. The modular system comprises an electronic module as described above. The modular system further comprises a drug delivery device as described above. Here, the mechanical coding of the electronic module matches with the mechanical coding of the drug delivery device. Moreover, the mechanical coding mechanically engages with the mechanical counter coding when the electronic module and the drug delivery device are in the predefined fastening configuration.

The modular system may include a number of differently configured electronic modules as well as a number of differently configured drug delivery devices. The numerous electronic modules may distinguish by their mechanical code or coding. The numerous drug delivery devices may distinguish by their mechanical counter code or coding. If an electronic module provided with a first mechanical coding should be subject to an assembly procedure with a drug delivery device provided with a second mechanical counter code not suitable for pairing or for engaging with the first mechanical coding, the first mechanical coding and the second mechanical counter coding, hence a mechanical coding non-matching with a mechanical counter coding, effectively prevent to establish the predefined fastening configuration of the electronic module at the drug delivery device.

Generally, the electronic module may comprise at least one of:
a battery or rechargeable accumulator holder, and/or
optionally a battery or a rechargeable accumulator, and/or
optionally only one or at least one circuit board, and/or
electronic parts, e.g. resistors and/or at least one integrated circuit, may form an electronic circuit or circuitry,
the electronic parts may comprise at least one sensor element, for instance an optical sensor, and/or
a microprocessor or microcontroller or another control unit, and/or
optionally a receiving and/or transmitting (sending) unit, for instance based on the Bluetooth protocol (may be a registered trade mark), the WiFi protocol (may be a registered trade mark) or on the USB protocol (Universal Serial Bus—may be a registered trade mark), for instance for communication with a smartphone or other computer device, and/or
an extension for the sensor, for instance a light guide that is for instance attached to a circuit board, and/or
at least one switch, for instance for power management.

The distal end of the electronic module and the proximal end of the drug delivery device may be arranged on a longitudinal axis of the drug delivery device. The second module may be arranged on the extended longitudinal axis of the drug delivery device. Thus, a serial coupling along the longitudinal axis may be realized.

Generally, the scope of the present disclosure is defined by the content of the claims. The injection device is not limited to specific embodiments or examples but comprises any combination of elements of different embodiments or examples. Insofar, the present disclosure covers any combination of claims and any technically feasible combination of the features disclosed in connection with different examples or embodiments.

In the present context the term ‘distal’ or ‘distal end’ relates to an end of the injection device that faces towards an injection site of a person or of an animal. The term ‘proximal’ or ‘proximal end’ relates to an opposite end of the injection device, which is furthest away from an injection site of a person or of an animal.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide. Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N-(N-lithocholyl-gamma-glutamyl)-des(B30) human insulin; B29-N-(ω-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091, MAR-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrom.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present invention include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen. Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present invention, which encompass such modifications and any and all equivalents thereof.

It will be further apparent to those skilled in the art that various modifications and variations can be made to the present disclosure without departing from the scope of the disclosure. Further, it is to be noted, that any reference numerals used in the appended claims are not to be construed as limiting the scope of the disclosure.

### BRIEF DESCRIPTION OF THE FIGURES

In the following, numerous examples of the injection device and the method of pairing a data logging device with an external electronic device will be described in greater detail by making reference to the drawings, in which:

FIG. 1 shows an example of a drug delivery device provided with an electronic module,

FIG. 2 schematically shows numerous components of an example of an electronic module,

FIG. 3 shows one example of a mechanical coding of an electronic module and of a correspondingly-shaped mechanical counter coding at the proximal end of the drug delivery device,

FIG. 4 shows another example of a mechanical coding of the electronic module configured to engage with a complementary-shaped mechanical counter coding provided at the proximal end of the drug delivery device,

FIG. 5 schematically illustrates a logical structure of an electronic unit of the electronic module,

FIG. 6 shows another example of an electronic module as seen from its distal side,

FIG. 7 shows the electronic module or its housing connected to a component of the drug delivery device,

FIG. 8 shows a cross-section through the assembly of FIG. 7,

FIG. 9 shows a top view of the drug delivery device with a first mechanical counter coding,

FIG. 10 shows another example of a drug delivery device with a second mechanical counter coding,

FIG. 11 shows a further example of an electronic module provided with a mechanical coding,

FIG. 12 shows an enlarged section of the electronic module of FIG. 11,

FIG. 13 shows another example of an interface of an electronic module and a drug delivery device in accordance to FIGS. 11 and 12,

FIG. 14 shows another example of a coded interface compared to FIG. 13,

FIG. 15 shows another example of a coded interface between the electronic module and the drug delivery device,

FIG. 16 shows another example of a coded interface between the electronic module and the drug delivery device,

FIG. 17 shows another example of a coded interface between the electronic module and the drug delivery device,

FIG. 18 shows another example of a coded interface between the electronic module and the drug delivery device.

### DETAILED DESCRIPTION

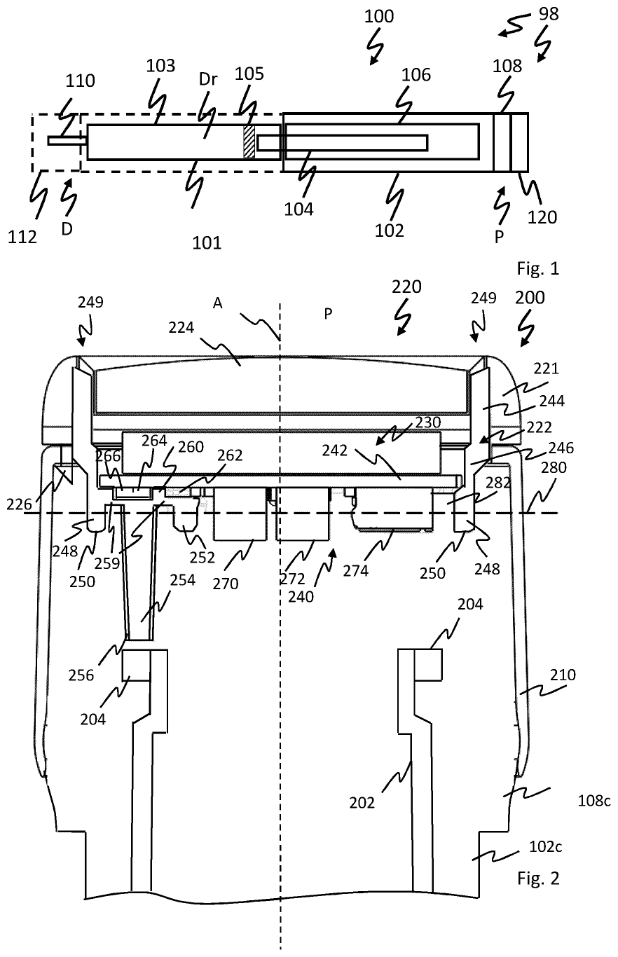

FIG. 1 illustrates a modular system 98 according to a first example. Modular system 98 may comprise a drug delivery device 100 that may comprise a container retaining member 101 and a main housing part 102. Container retaining member 101 may accommodate a drug container 103. The drug container 103 may comprise a cartridge sealed in proximal direction by a movable stopper 105 the drug container 103 may comprise a drug Dr. The main housing part 102 may house or surround the container retaining member 101 completely or partially and may comprise further parts of the drug delivery device 100. Alternatively, the main housing part 102 may be connected to the container retaining member 101 but may not surround it and even may not surround a part of the container retaining member 101, see dashed line in FIG. 1.

Within the main housing part 102 the following components may be arranged:
a piston rod 104 that is adapted to move the piston that may be arranged within container retaining member 101,
a driving mechanism 106 for the piston rod 104. The driving mechanism 106 may comprise an energy storing element, for instance a spring that is loaded manually before each use. Alternatively, the energy storing element may be loaded for instance during assembling of drug delivery device 100. Alternatively, a manually driven driving mechanism may be used, e.g. without an energy storing element that is used to drive piston rod 104.

With some examples and for instance at a proximal end P, an actuating element 108 is arranged that is used for the initiation of a movement of piston rod 104 into the container retaining member 101, whereby the driving mechanism 106 is used. Alternatively, an autoinjector device may be used that is actuated by an axial movement of a movable needle shroud (not shown). Actuating element may be used to dial the size of a dose of drug Dr in some embodiments.

Also, a cap 112 may be attached to main housing part 102 or to another part of drug delivery device 100. Cap 112 may be an outer cap that may include a smaller inner cap which protects a needle 110 directly.

If drug delivery device 100 is not an autoinjector, a dial sleeve may be screwed out of main housing 102 and may be pressed by a user in order to move plunger 104 distally and to inject drug Dr.

Drug delivery device 100 may be a single use or a multiple use device.

Drug Dr may be dispensed from the container through needle 110 or through a nozzle that is connectable and/or connected to the distal end D of drug delivery device 100. Needle 110 may be changed before each use or may be used several times.

Modular system 98 may comprise an electronic module 120 that is mechanically connected to a proximal end region P of drug delivery device 100, for instance to a proximal end region P of actuating element 108. Modular system 98 is described below in more detail, see FIG. 2 and corresponding description.

Electronic module 120 may be used not only for drug delivery device 100 but also for other drug delivery devices that are similar or identical to drug delivery device 100. Thus, electronic module 120 is used multiple times with different drug delivery devices in different modular systems 98, etc. Furthermore, the diameter of drug delivery device 100 is not increased by electronic module 120 promoting excellent handling of modular system 98, and especially of drug delivery device 100.

FIG. 2 illustrates a modular system 200 of a second embodiment that may be the same as the first embodiment. However, more details are shown in FIG. 2. Modular system 200 may comprise for instance a housing part 102c that may correspond to housing part 102 described above. The actuating element 108c may correspond to actuating element 108 described above.

The modular system 200 may comprise:
a clutch element 202 or other rotatable or moving element that may comprise radially protruding features 204, for instance teeth of a sprocket or of a sprocket sleeve, e.g. providing a rotary encoder,
an essentially annular adapter element 210 that may encompass the side wall of actuating element 108,
an electronic module 220 that may correspond to electronic module 120 and that may comprise an electronic unit 240. Electronic unit 240 is described below in more detail.
an annular casing or housing 221 of electronic module 220,
a chassis 222 within electronic module 220. Chassis 222 may comprise an annular wall 249 surrounding a compartment for electronic unit 240 and/or for several other parts.
and a lid 224 of electronic module 220.

Fastening elements 226 may be used to connect housing 221 and adapter element 210. Alternatively, other connection means may be used or housing 221 and adapter element 210 may be formed integrally as one single part.

The following electronic components may be comprised within electronic module 220:
a battery 230 or a rechargeable accumulator, and
an electronic unit 240 that may form a PCBA (Printed Circuit Board Assembly).

Electronic unit 240 may comprise:
a printed circuit board 242 (PCB) which may be named as substrate in the claims,
at least one light source 264, e.g. an IR (Infra-Red) light source, or two light sources,
at least one optical sensor 266 or at least two optical sensors,
a transmitter unit 270, for instance a transmitter unit 270 that operates according to the Bluetooth (may be a registered trademark) protocol, for instance for communication with a smartphone or other computer device,
a receiver unit 272, for instance a receiver unit 272 that operates according to the Bluetooth (may be a registered trademark) protocol, for instance for communication with a smartphone or other computer device, and
an optional switch 274, for instance a micro switch.

FIG. 2 shows a longitudinal axis A of modular system 200. Electronic module 220 may be arranged proximally of actuating element 108c of the corresponding drug delivery device. Electronic module 220 and actuating element 108c are arranged symmetrically to axis A whereby electronic module 220 and actuating element 108c are in physical contact with each other, mainly via adapter element 210. Adapter element 210 may be plugged mechanically onto actuating element 108c.

The chassis 222 may comprise:
three annular wall portions 244, 246 and 248 of an annular wall 249,
a distal end 250 of chassis 222 and at the same time of annular wall portion 248,
a wall 252 of chassis 222, and
at least one optical guide 254 or at least two optical guides 254, 258.

A cup like structure may be formed by wall 252 and by a part of annular wall portion 248 around a proximal part or base part of optical guide 254. The cup like structure may comprise a laterally extending thinner portion 259 that may be regarded as a bottom portion of the cup like structure. Thinner portion 259 may be arranged near to but distally of a light source 264, e.g. IR, and of an optical sensor 266. A rib 260 may be arranged on thinner portion 259 and may extend proximally P up to printed circuit board 242. Rib 260 may be adjacent to light source 264, e.g. IR, and/or optical sensor 266. There may be a gap 262 between printed circuit board 242 and thinner portion 259 and/or a proximal or bottom portion of wall 252. Gap 262 may be filled with a potting compound/material 282. Rib 260 may protect light source 264, e.g. IR, and/or optical sensor 266 against potting compound/material 282 if it is in its melted state.

There may be a sequence of annular wall portions 244, 246 and 248 in this order from proximal P end to distal end of annular wall 249. Annular wall portion 244 may have a first diameter that corresponds to the diameter of the lid 224. The annular wall portion 246 may have a second diameter that is less than the first diameter. The second diameter may correspond to the diameter of printed circuit board 242. Furthermore, annular wall portion 248 may have a third diameter that is less than the second diameter.

A fill height 280 measured from PCB 242 may be in the range of 2 mm to 7 mm. The fill height 280 of the potting compound 282 or material may be selected appropriately, for instance to cover only a part of some of the electrical parts of electronic unit 240. The inner side of distal end 250 of annular wall portion 248 may not be covered by potting compound 282 or by another potting material. The potting compound 282 or potting material may be an electrical insulator. The wall 252 may protect the basis part of optical guide 254 against the potting compound/material 282 during potting.

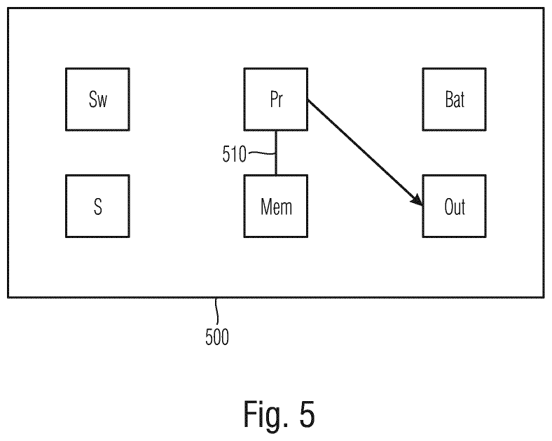

FIG. 5 illustrates schematically an electronic unit 500, for instance electronic unit 240. The electronic unit 500 may comprise:
at least one processor Pr or another control unit,
a memory Mem, for instance volatile and/or nonvolatile storing memory,
a battery Bat or a rechargeable accumulator or any other electrical power source,
an output device Out, for instance a sending unit, for instance for communication with a smartphone or other computer device,
an optional input device In, for instance for communication with a smartphone or other computer device,
a switch Sw, and
at least one sensor S or at least two sensors, preferably optical sensor(s).

Further parts may be comprised in electronic unit 500 that are not shown, for instance a radiation source, especially a light source.

Processor Pr may be a microcontroller or microprocessor that performs instructions of a program which is stored in memory M. Alternatively, an FPGA (Field Programmable Gate Array), ASIC (Application Specific Integrated Circuit), PLA (Programmable Logic Array), PLD (Programmable Logic Device) or another appropriate circuitry may be used to implement a finite state machine that does not perform instructions of a program.

The electronic unit 500 may implement a quadrature encoder, e.g. an encoder that uses amplitude modulation 180 of two sensors having 180 degree phase shift between two sensor signals, for instance anti phase sensor signals. Alternatively, other sensing methods may be used.

There may be two alternative modes of operation of sensing in accordance with various embodiments. According to a first alternative, a first sensor and a second sensor, for instance optical sensors, may be provided having an angular offset that is half of the periodicity of encoded regions of the encoder ring, for instance on clutch element 202. In the embodiment according to the first alternative, the sensors may be operated to sample synchronously, i.e. at the same times (t1; t2, t3, . . . ). This may ease signal detection and/or signal processing.

According to a second alternative, a first sensor and a second sensor, for instance optical sensors, may be provided having an angular offset that differs from half of the feature periodicity of encoded regions of the encoder ring. Therefore, sensors I and II may operate in a staggered mode with an offset in time (delta t) between samplings. This may be used to achieve more balanced overall system power consumption than available in synchronous operation.

One of the following sensing modes may be used:
1) static thresholding,
2) dynamic thresholding,
3) not using a threshold to detect low-high transitions of the sensor signals. However, a threshold for a voltage difference of the two sensor signals may be used. Furthermore, scaling factors for mean and amplitude may be used. The scaling factors may be set during manufacturing, for instance during a calibration method.
4) same as 3) but differing in that the scaling factors may be calculated after each dose delivery. 5) peak-detect method that does preferably not use the setting of thresholds to detect low-high transitions of the sensor signal(s) and that does preferably not use scaling of signals to match mean and amplitude.

Spoken with other words one part of the disclosure relates to light pipe/guide 254 or optical pipe protection, preferably against loads. The optical pipe may be an optical fiber, a tube or other optical guiding means. An additional coating 256 on the outer surface of the light pipe may be used to prevent damage from loads coming from outside to the light pipe. One option would be a metal coating or a similar robust material coating to stiffen up the structure of the light pipe. A second option would be a soft coating to absorb impact loads resulting in less stresses of the light pipe. Another option would be reinforced coatings e.g. carbon fiber reinforced polymer (in German language: CFK) filled materials. A combination of two or of three of these options is possible as well.

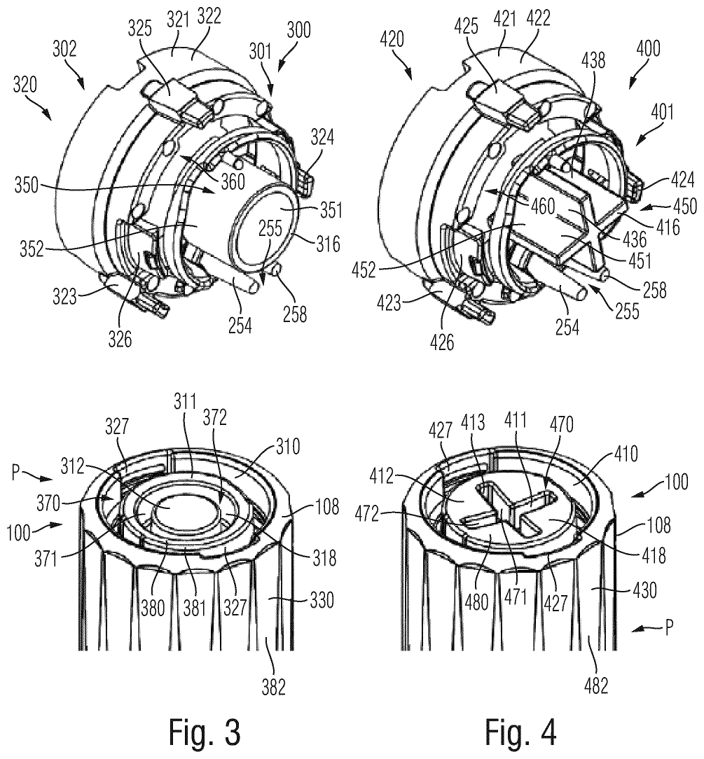

FIG. 3 illustrates another example of a modular system 300. Generally, the modular system 300 is similar or almost identical to the modular system 98 or 200 as described above in connection with FIGS. 1 and 2. The modular system 300 comprises an electronic module 320. The electronic module 320 comprises a housing 321. The housing 321 may form or comprise a chassis 322. The chassis 322 is somewhat identical or equivalent to the chassis 222 as described above in connection with FIG. 2.

The electronic module 320 comprises a distal end 301 and a proximal end 302. Near or at the distal end 301 the electronic module 320 comprises a distally facing surface 360. At the distal surface 360 and/or at the distal end 301 there is further provided a mechanical coding 350. The mechanical coding 350 comprises a longitudinally extending protrusion 352 forming or constituting a mechanical coding feature 351. In the present example, the mechanical coding feature 351 comprises an annual shaped or tubular shaped hollow sleeve.

Along the circumference of the housing 321 there are provided numerous fastening elements 323, 324 and 325, 326. The fastening elements 323, 324, 325 may be arranged equidistantly along the circumference of the housing 321. Moreover, there are provided two separate optical guides, e.g. in form of light guides or light pipes 254, 258 also protruding distally from the distally facing surface 360 at the distal end 301. In FIG. 3 there is further illustrated a proximal end P of the drug delivery device 100. The proximal end P may comprise the actuating element 108. Thus, the movable, e.g. rotatable actuating element 108 may constitute or may form the proximal end P of the drug delivery device 100.

At the proximal end P there is provided a mechanical counter coding 370. The mechanical counter coding 370 comprises a mechanical counter coding feature 371. Here, the mechanical counter coding feature 371 comprises an annular recess 372 configured to engage and/or to receive the protrusion 352 and hence the mechanical coding feature 351 of the electronic module 320. The recess 372 may be located radially between a central cylindrical portion 312 and a surrounding sidewall portion featuring a proximally facing surface 380. The proximally facing surface of the cylindrical portion 312 and the surface 380 as well as a proximal end of the actuating element 108 may flush and may be located in a common transverse plane.

The surface 380 is provided on an inner annular shaped sidewall 381 or annular ring 311. Between the inner sidewall 381 and an outer side wall 382 of the actuating element 108 there is provided an annular groove 310. Radially between the inner sidewall 381 and the outer sidewall 382 there is provided an outer annular groove 310 to receive the optical guides 254, 258. The outer annular groove 310 is separated by the annular ring 311 and hence by the inner sidewall 381 from the annular recess 372.

The optical guides 254, 258 extend into the annular groove 310 when the electronic module 320 and hence it chassis 322 is mounted or assembled to the drug delivery device 100 in at least one of two available predefined fastening configurations. The optical guides 254, 258 may extend the same length as the keying feature 316 or coding feature 351 measured for instance from a circuit board within the chassis 322. Alternatively, the optical guides 254, 258 may be slightly shorter than the mechanical coding feature 351. With some examples, only one of the optical guides 254 or 258 may be used.

The recess 372, hence the annular groove forming or constituting the keying feature 318 is complementary shaped to the keying feature 316. In effect, only a drug delivery device 100 equipped with the mechanical counter coding feature 371 can be connected with the presently illustrated electronic module 320 provided with the complementary shaped coding feature 351. Insofar, the keying feature 318 and hence the mechanical counter coding feature 371 is inverse shaped compared to the keying feature 316 or mechanical coding feature 351. The counter coding feature 371 comprises an inner diameter sized and configured to receive the outer dimensions of the mechanical coding feature 351.

The longitudinal extent of the mechanical counter coding feature 371 is larger than or equal to the longitudinal extent of the complementary shaped coding feature 351. This allows and supports a complete insertion of the coding feature 351, e.g. of the protrusion 352, into the complementary-shaped counter coding feature 371, e.g. into the recess 372.

With other, non-matching examples, the longitudinal extent of the coding feature 351 may be larger than a corresponding longitudinal extent of the counter coding feature 371. In this way, and when the coding feature 351 enters or engages the counter coding feature 371 the entirety of the coding feature 351 cannot be received in the counter coding feature 371 thereby preventing a mutual engagement of the fastening elements 326 with the complementary-shaped counter fastening elements 327. Here, an insert motion of the coding feature 351 into the counter coding feature 371 is effectively blocked, e.g. by a blocking feature or an end wall of the counter coding feature 371. Accordingly, and when reaching such a blocking configuration the fastening elements 326 will be located at a longitudinal offset from the counter fastening elements 327. Here, a fastening of the electronic module 320 to the drug delivery device 100 will be effectively prevented.

The same applies when the geometric shape of the protrusion 352 of the coding feature 351 does not match with the geometric shape of the recess 372 of the counter coding feature 371 or when a position of the protrusion 352 in the plane transverse to the longitudinal direction is not matching with the respective position of the recess 372 in the transverse plane.

The outer sidewall 382 of the actuating or adjusting element 108 comprises numerous counter fastening elements 327 on its inside facing sidewall portion and hence towards the annular groove 310. The counter fastening elements 327 are configured to engage with correspondingly shaped fastening elements 326 of the electronic module 320. The fastening elements 326 protrude distally from the distal face 360 of the housing 321. The fastening elements 326 and/or the counter fastening elements 327 are configured as snap elements or clip elements and they are hence configured to form a snap fit or clipped connection between the electronic module 320 and the drug delivery device 100.

The further fastening elements 323, 324, 325 may comprise or may form a hook, each of which being configured to engage with a longitudinal groove 330 on the outside surface of the actuating/adjusting element 108.

The hooks or fastening elements 323, 324, 325 serve to block a relative rotation between the electronic module 320 and the actuating/adjusting element 108. The hooks or fastening elements 323, 324, 325 cooperate with the grooves 330. Alternatively, an adapter element may be used that corresponds to the adapter element 210 as illustrated in FIG. 2. The hooks or fastening element 323, 324, 325 may provide or contribute to an axial fixing of the electronic module 322 to the drug delivery device 100, e.g. by a clamping or friction fit to the actuating/adjusting element 108.

In an alternative but not illustrated example, the mechanical coding feature 351 and hence the keying feature 316 may be arranged at the actuating/adjusting element 108, whereas the annular ring 311 is located on the chassis 322 or housing 321 of the electronic module 320.

The fastening elements 326 and the counter fastening elements 327 may form or constitute a clip connection and may provide a detachable fastening of the electronic module 322 from the drug delivery device 100.

The further example of a modular system 400 as illustrated in FIG. 4 comprises a similar structure but distinguishes from the electronic module 320 as shown in FIG. 3 by the specific implementation of the mechanical coding 450 and the mechanical coding feature 451. Accordingly, also the mechanical counter coding 470 and the respective mechanical counter coding feature 471 of the drug delivery device 100 distinguish from the example of FIG. 3.

As already described in connection with FIG. 3, the electronic module 420 comprises two diametrically oppositely located fastening elements 426 to engage with correspondingly shaped counter fastening elements 427 on an inside of the outer sidewall 482 of the actuating/adjusting element 108. The housing 421 and/or the chassis 422 of the electronic module 420 further comprises a number of supplemental fastening element 423, 424, 425. These fastening elements 423, 424, 425 may be implemented as a hook to engage with longitudinal grooves 430 on the outside surface of the actuating/adjusting element 180 when the electronic module 420 is assembled and/or attached to the proximal end P of the drug delivery device 100 in the predefined fastening configuration.

The housing 421 or the chassis 422 comprises a distally facing surface 460. The optical guides 254, 258 as well as the numerous fastening elements 423, 424, 425, 426 may protrude distally from this distally facing surface 460. The distal end 401 of the chassis 422 is provided with a mechanical coding 450. The mechanical coding 450 comprises a mechanical coding feature 451. The mechanical coding feature 451 comprises a geometric structure with cross-shaped cross section protruding distally from the distally facing surface 460. It may provide a keying feature 416. The proximal end P of the drug delivery device 100, hence the proximal surface 480 of the actuating/adjusting member element 108 comprises a complementary-shaped counter coding feature 471. The counter coding feature 471 forms a mechanical counter coding 470 matching with the mechanical coding 450 when the electronic module 420 is assembled and attached to the drug delivery device 100 in the predefined fastening configuration.

The counter coding feature 471 comprises a recess 472. The recess is shaped in a cross wise manner. The recess 472 is provided in the central cylindrical portion 412 of the actuating/adjusting element 108. The recess 472 comprises two elongated mutually crossing slits 411, 413. Here, the slit 411 may be shaped to receive a slab—of plate-like element 438 of the coding feature 451. The slit 413 may be shaped and arranged to engage and/or to receive the further slab—or a plate-like element 436 of the mechanical coding feature 451.

Between the central cylindrical portion 412 and the outside or outer sidewall 482 there is also provided an annular groove 410 to receive the light guides 254, 258.

It should be noted, that the light guides 254, 258 are an example of a longitudinal extension 255 that may be provided on the chassis 322, 422 of the electronic modules 320, 420 as a symmetry breaking feature.

Generally, the codings 350, 450 may be defined by the shape of the mechanical coding features 351, 451 as well as by the position of the mechanical coding features 351, 451 relative to at least one of the extension 255 and/or relative to at least one of the fastening elements 326, 426.

When the electronic module 320, 420 is in the predefined fastening configuration on the drug delivery device 100, the distally facing surface surface 360, 460 of the electronic module 320. 420 may be in abutment with the proximally facing surface 380, 480 provided at the proximal end P of the drug delivery device 100. In this way a rather tilt-free mechanical fastening of the electronic module 320, 420 to the drug delivery device 100 can be provided.

The cross-like and non-rotationally symmetric structure of the coding feature 451 and the respective counter coding feature 471 is further beneficial to provide a torque proof engagement between the electronic module 420 and the actuating/adjusting element 108. The adjusting element 108 may be rotatable, e.g. for adjusting or setting of a dose of the drug. Here, the user may use the electronic module 420 as a kind of a dial extension. The user may use or may grip the chassis 422 or the housing 421 of the electronic module 420 to induce a respective torque onto the actuating/adjusting element 108.

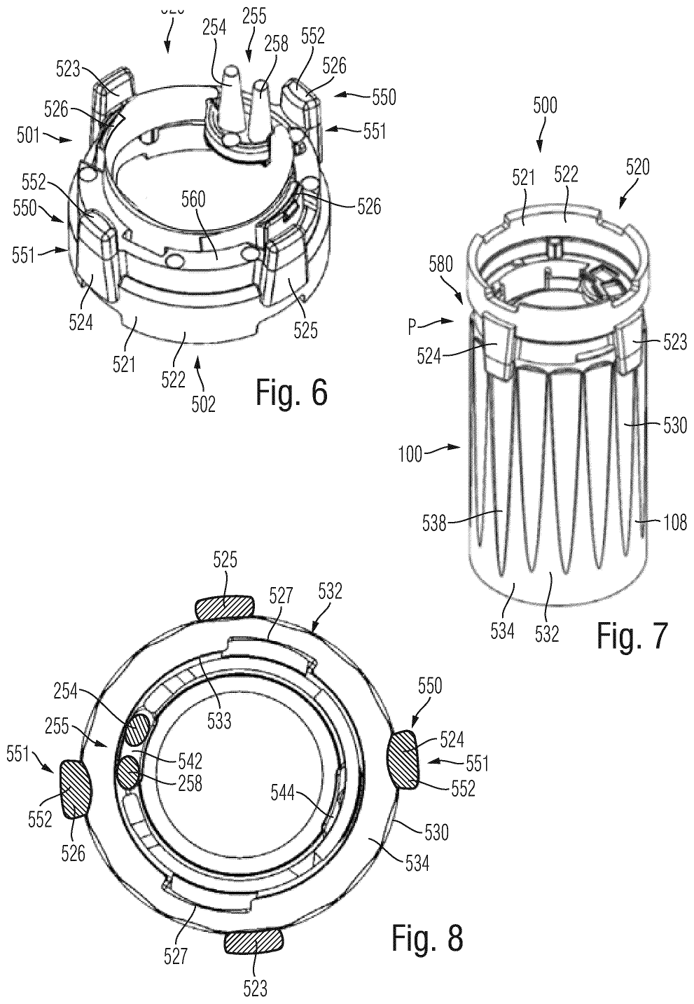

In the sequence of FIGS. 6 to 10 there is shown another example of an electronic module 520 suitable for attachment to a proximal end P of a drug delivery device 100. Again, the electronic module 520 is configured for attachment to an actuating/adjusting element 108 as shown in FIG. 2. The electronic module 520 comprises a housing 521 with a chassis 522. The housing 521 comprises a distal end 501 and a proximal end 502. The housing 521 or the chassis 522 may comprise a somewhat tubular shaped structure. Here, a distally facing surface 560 is provided on a circumferential rim of the housing facing in distal direction and being located at or near a distal end 501 of the housing 521.

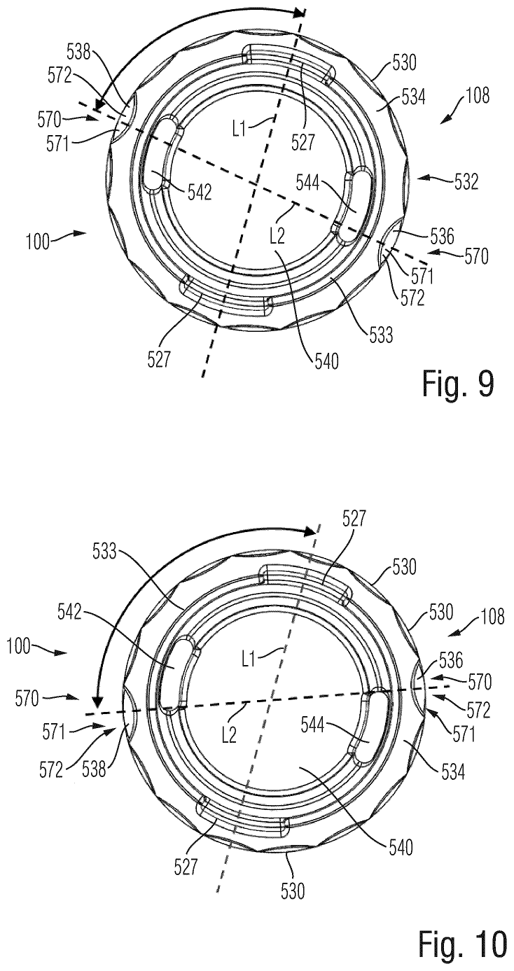

As described before in connection with FIGS. 3 and 4, also with the example of FIGS. 6 to 10 there are provided numerous fastening elements 523, 524, 525, 526 around the outer circumference of the housing 521. The fastening elements 523, 524, 525, 526 may protrude from the distal end 501 of the housing 521. They may protrude at least from the distally facing surface 560. As it is apparent from FIGS. 8 and 9 at least two of the fastening elements, namely the fastening elements 524 and 526 serve to provide a mechanical coding 550.

The mechanical coding 550 comprises at least one coding feature 551, 552. Here, the coding feature 551 coincides with the fastening element 526. In other words, the mechanical coding feature 552 is formed by the fastening element 526. The other coding feature 551 is provided and/or formed by the fastening element 524.

On or near the distal end 501 of the housing 521 there is further provided at least one supplemental fastening element 526. The fastening element 526 is configured to engage with a correspondingly or complementary-shaped counter fastening element 527 provided at an inside surface of a sidewall 534 of the actuating/adjusting element 108.

On the outside surface 532 of the sidewall 534 of the actuating/adjusting element 108 there are provided numerous slots or grooves 530. In the present example, there is provided at least one coding groove 536, 538 featuring a radial depth that is larger than a radial depth of other grooves 530 provided on the outside surface 532. The coding grooves 536, 538 are configured to exclusively engage with the mechanical coding features 551, 552 of the mechanical coding 550 of the electronic module 520. The radial depth as well as the circumferential or tangential size of the coding grooves 536, 538 matches with the respective geometric shape of the protrusions 552, forming the mechanical coding feature. The protrusions 552 may comprise a radially inwardly extending engaging structure complementary shaped to the geometry and dimensions of the coding grooves 536, 538.

As it is further apparent from FIG. 8, the actuating/adjusting element 108 further comprises two apertures 542, 544 that are diametrically opposite to each other adjacent to the inside 533 of the sidewall 534. The apertures 542, 544 are shaped and configured to receive the optical guides 254, 258 and hence the longitudinal extension 255 protruding from the distal end 501 of the electronic module 520. The position of the mechanical coding features 551 relative to the longitudinal extension 255 defines the mechanical coding 550 of the electronic module 520.

Accordingly, and as it becomes apparent from a comparison of FIGS. 9 and 10, the position of the coding grooves 536, 538 relative to the apertures 542, 544 and/or relative to the counter fastening features 527 defines a respective counter coding 570. A mutual assembly of the electronic module 520 and the drug delivery device 100 is only possible when the longitudinal extension 255 is in alignment with one of the apertures 542, 544. Moreover, a mutual assembly is only possible and when the fastening elements 526 are longitudinally aligned with the counter fastening elements 527. As such, the mutual orientation and the alignment of the fastening elements 526 with the counter fastening elements 527 and/or the alignment of the longitudinal extension 255 with the aperture 542, 544 define two specific predefined fastening configurations mutually distinguish by a relative rotation of the 180°.

In FIG. 9 there is illustrated a virtual L1 extending through the middle of the oppositely located counter fastening elements 527. There is further shown another virtual line L2 extending through the diametrically oppositely located mechanical counter coding features 571.

In the example of FIG. 10 there is shown a mechanical counter coding 570 that distinguishes from the mechanical counter coding 570 as illustrated in FIG. 9. With the example of FIG. 10, the angle between the respective L1 and L2 distinguishes from the respective angle of the example of FIG. 9. In the example of FIG. 9, the angular position or the tangential or circumferential position of the counter coding features 571 relative to the counter fastening elements 527 and/or relative to the apertures 542, 544 has changed compared to the configuration of FIG. 9. Accordingly, the proximal end of the drug delivery device 100 as illustrated in FIG. 10 comprises a mechanical counter coding that distinguishes from a mechanical counter coding at a proximal end of a drug delivery device 100 as illustrated in FIG. 9.

The coding grooves 536, 538 are presently configured at a radial recess 572 in the outside surface 532 of a sidewall 534 of the actuating/adjusting element 108. By varying the angular position of the mechanical counter coding features 571 relative to the fixed position of the counter fastening element 527 and/or relative to the position of the apertures 542, 544 numerous different mechanical counter codings 570 can be provided for generally equal or similar types of drug delivery devices 110. In the same way, also the position of the coding features 551, and in particular of the fastening elements 524, 526 may be subject to variations to provide different mechanical codings for numerous electronic modules 520.

In case that an electronic module 520 comprising a mechanical coding 550 non-matching with the mechanical counter coding 570 of the drug delivery device 100, the coding features 551 will be located circumferentially offset and hence outside the coding grooves 536, 538. In the predefined fastening configuration, in which the fastening elements 526 engages with the correspondingly shaped counter fastening element 527, the coding features 551, hence the protrusions 552 may have no suitable channel to run in. They may thus block and impede a proper arrangement and assembly of the electronic module 522 the drug delivery device 100.

As seen in FIGS. 9 and 10, the coding grooves 536 may end in a radially outwardly extending flange provided at the very proximal end P of the actuating/adjusting element 108. In this way, the coding grooves 536, 538 and hence the respective counter coding features 571 may provide a snap fit engagement with the correspondingly-shaped protrusions 552 of the respective coding features 551 of the mechanical coding 550 of the electronic module 520.

The mechanical coding features 551 may each comprise a resiliently deformable snap feature to form a snap fit connection with the correspondingly shaped counter coding features 571 provided at the proximal end P of the drug delivery device 100. The snap fit engagement may further provide a longitudinal fastening and/or fixing between the electronic module 320 and the drug delivery device 100.

The proximal end P of the drug delivery device 100 may also comprise a proximally facing surface 580, which is indicated in FIG. 7. In the predefined fastening configuration as shown in FIG. 7, the distally facing surface 560 of the electronic module 520 is in abutment with the proximally facing surface 580 of the actuating/adjusting element 108. In this way, a tilt-free mutual fastening between the electronic module 520 and the drug delivery device 100 can be provided.

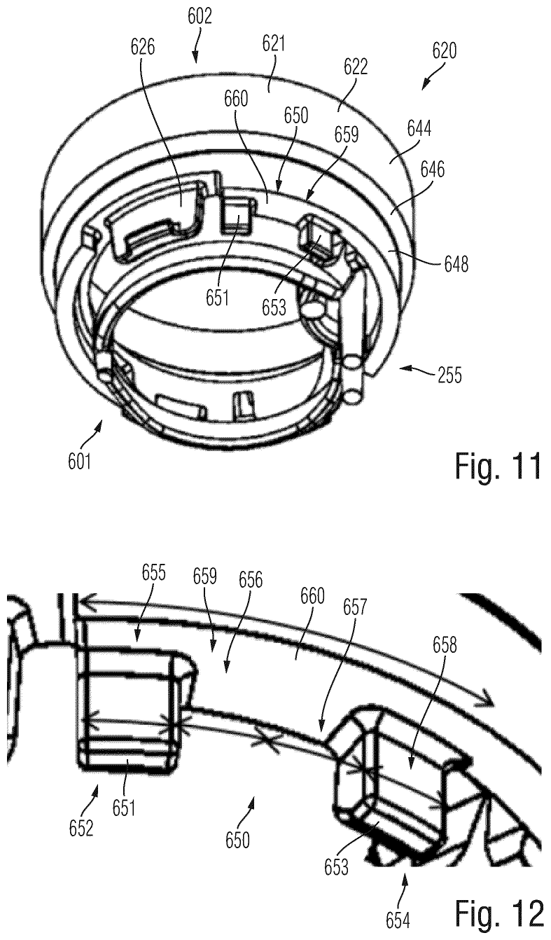

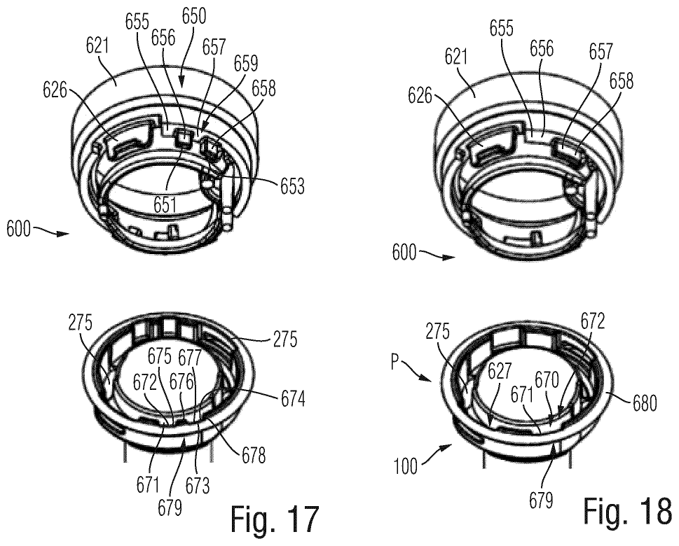

In the further example as illustrated in FIGS. 11-18 the electronic module 620 comprises a housing 621 with a chassis 622. The electronic module 620 comprises a distal end 601 and an oppositely located proximal end 602. The electronic module 620 may be somewhat equivalent or even identical to the electronic module 220, 320, 420 or 520 as described above in connection with FIG. 2, 3, 4 or 6, respectively. The electronic module 620 distinguishes from the further examples by a particular type of a mechanical coding 650. The housing 621 comprises numerous wall portions 644, 646, 648 that are somewhat equivalent or identical to the wall portions 244, 246, 248 as described above.

A distal end of the wall portion 648 is provided with a distally facing surface 660. On this surface 660 there is provided a mechanical coding section 659. Apart from that, the housing 621 comprises a fastening element 626 implemented as a snap feature or as a clip feature to engage with a correspondingly shaped counter fastening element 627 provided on the proximal end P of the drug delivery device 100. The proximal end P of the drug delivery device 100 is provided with a proximally facing surface 680. When the electronic module 620 is in the predefined fastening configuration the proximally facing surface 680 may abut with the distally facing surface 660.

With the examples of FIGS. 11-18, the mechanical counter coding 670 may be provided on a portion of an actuating/adjusting element 108. It may be provided on a radial inner part of the actuating/adjusting element 108. Hence, the actuating/adjusting element 108 may comprise such an inner part and an outer, e.g. sleeve-shaped part surrounding the inner part. The outer part may comprise an outside wall 534 as for instance illustrated in the example of FIG. 7.

In the present example the coding section 659 comprises four discrete spatially non-overlapping coding feature positions 655, 656, 657, 658 as illustrated in greater detail in FIG. 12. The coding feature positions 655, 656, 657, 658 may be individually provided with a coding feature 651, 653. In the presently illustrated example, there are provided two individual coding features 651, 653. The coding feature 651 is provided on the first coding feature position 655. The second coding feature 653 is provided on a fourth coding feature position 658. The coding feature positions 656, 657 are void of a coding feature. The coding features 651, 653 each comprise a protrusion 652, 654 protruding in distal direction from the distally facing surface 660. The protrusion 652, 654 of the coding features 651, 653 comprise a tab-like geometry. The coding features 651, 653 are complementary shaped and/or complementary positioned to counter coding features 671, 673 provided on the proximal end P of the drug delivery device 100 as illustrated for instance in FIG. 15.

There, the counter coding 670 comprises a complementary-shaped counter coding section 679. The counter coding section 679 comprises a number of discrete spatially non-overlapping counter coding feature position 675, 676, 677, 678. In the example of FIG. 14, a counter coding feature 671 in the form of a recess 672 is provided on a first counter coding feature position 675 and another coding feature 673 in form of another recess 674 is provided on a third counter coding feature position 677.

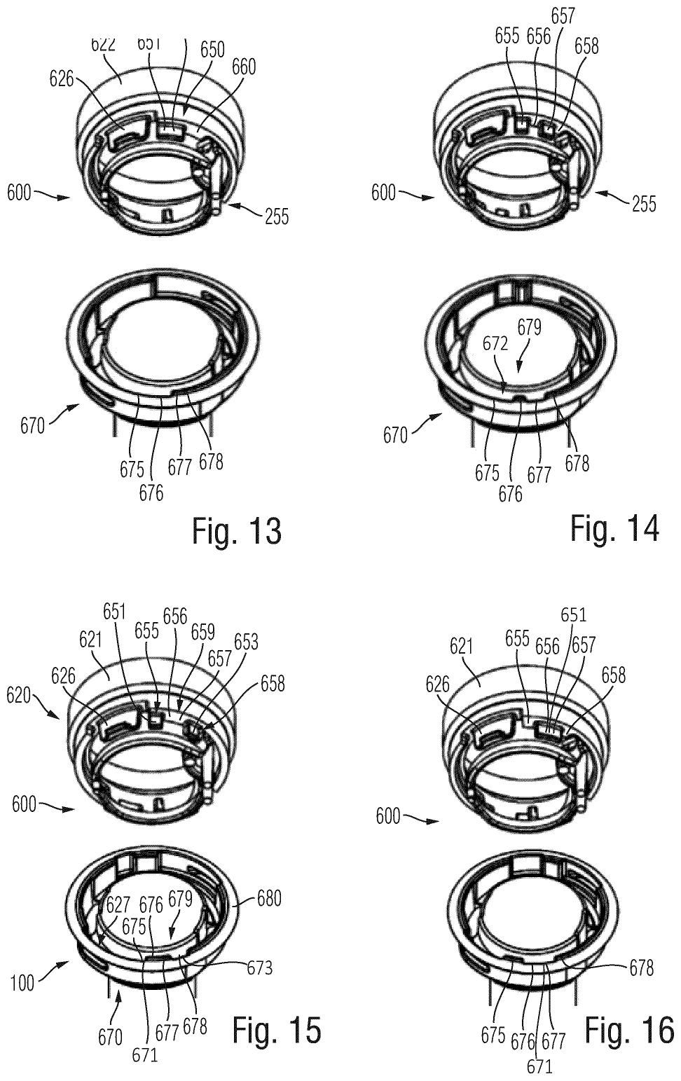

As it is apparent with all examples of FIGS. 13 and 18, the mechanical coding 650 is complementary shaped to a respective mechanical counter coding 670. The mechanical counter coding 670 is of inverse shape compared to the respective mechanical coding 650.

In the present examples, the coding section 659 comprises four discrete spatially non-overlapping coding feature positions for two mechanical coding features, each one of which located on one of the coding feature positions. Likewise, the mechanical counter coding section 679 comprises four discrete spatially non-overlapping counter coding feature positions 675, 676, 677, 678 and two mechanical counter coding features 671, 673 each one of which located on one of the counter coding feature positions.

For the examples as shown in FIGS. 13-18 the number of coding feature positions of the coding section 659 equals the number of counter coding feature positions of the counter coding section 679. Moreover, the number of coding features equals the number of counter coding features. So with the presently illustrated example with four coding feature positions and with two mechanical coding features there can be provided six different mechanical codes or codings 650 and respective mechanical counter codes or codings 670. Generally, if the total number of discrete spatially non-overlapping coding feature positions is n, there are 2ⁿ possible configurations and arrangements. But not all of these arrangements may guarantee a unique and dedicated pairing. The condition to ensure that that all but one of the electronic modules 620 is hindered to engage with a drug delivery device is that every mechanical coding 650 comprises the same number of mechanical coding features 651, 653. The number of unique permutations of k coding features in n coding feature positions is equal to the binomial coefficient according to the following equation:

$$( \begin{matrix}n \\ k\end{matrix} ) = \frac{n !}{k ! ( n - k ) !}$$

The maximum number of available combinations is obtained when k=n/2.

1-15. (canceled)

16. An electronic module configured for attachment to a proximal end of a drug delivery device in a predefined fastening configuration, the drug delivery device comprising an elongated housing extending in a longitudinal direction and comprising a distal end and the proximal end, the electronic module comprising:
a mechanical coding comprising a mechanical coding feature to engage with a mechanical counter coding feature of a mechanical counter coding provided at the proximal end of the drug delivery device,
wherein one of the mechanical coding feature and the mechanical counter coding feature comprises a protrusion extending in the longitudinal direction, and wherein the other one of the mechanical coding feature and the mechanical counter coding feature comprises a recess, and
wherein when a geometric shape of the protrusion is not matching with a geometric shape of the recess, or when a position of the protrusion in a plane transverse to the longitudinal direction is not matching with a position of the recess in the transverse plane, or when a longitudinal extent of the protrusion is larger than a longitudinal extent of the recess, the mechanical coding and the mechanical counter coding being operable to prevent a fastening of the electronic module to the proximal end of the drug delivery device in a predefined fastening configuration.

17. The electronic module according to claim 16, further comprising a fastening element configured to mechanically engage with a complementary shaped counter fastening element of the drug delivery device in the predefined fastening configuration.

18. The electronic module according claim 17, wherein the mechanical coding is defined by at least one of a position, an orientation and a longitudinal extent of the mechanical coding feature relative to the fastening element.

19. The electronic module according to claim 16, further comprising at least one longitudinal extension extending distally from a distal end of the electronic module and configured to extend into or through an aperture at the proximal end of the drug delivery device.

20. The electronic module according to claim 19, wherein the mechanical coding is defined by at least one of a position, an orientation and a longitudinal extent of the mechanical coding feature relative to the longitudinal extension.

21. The electronic module according to claim 16, wherein the mechanical coding feature comprises the protrusion protruding in a longitudinal distal direction from a distally facing surface of the electronic module and configured to engage with a complementary shaped recess of the mechanical counter coding feature.

22. The electronic module according to claim 21, wherein the distally facing surface of the electronic module is in longitudinal abutment with a complementary shaped proximally facing surface of the drug delivery device.

23. The electronic module according to claim 16, wherein the mechanical coding comprises a coding section comprising a number of n discrete spatially non-overlapping coding feature positions and a number of k mechanical coding features each one of which located on one of the coding feature positions, wherein n and k are integer numbers and wherein k≤n.

24. A drug delivery device comprising:
a housing comprising a distal end and a proximal end, wherein the proximal end is configured for attachment of an electronic module in a predefined fastening configuration, wherein the electronic module comprises:
a mechanical coding comprising a mechanical coding feature to engage with a mechanical counter coding feature of a mechanical counter coding provided at the proximal end of the drug delivery device,
wherein one of the mechanical coding feature and the mechanical counter coding feature comprises a protrusion extending in the longitudinal direction, and wherein the other one of the mechanical coding feature and the mechanical counter coding feature comprises a recess, and
wherein when a geometric shape of the protrusion is not matching with a geometric shape of the recess, or when a position of the protrusion in a plane transverse to the longitudinal direction is not matching with a position of the recess in the transverse plane, or when a longitudinal extent of the protrusion is larger than a longitudinal extent of the recess, the mechanical coding and the mechanical counter coding being operable to prevent a fastening of the electronic module to the proximal end of the drug delivery device in a predefined fastening configuration, the drug delivery device comprising:
a drive mechanism configured to set and/or to deliver a dose of a drug out of the distal end; and
wherein the mechanical counter coding is provided at the proximal end and comprises the mechanical counter coding feature to engage with the mechanical coding feature of the mechanical coding of the electronic module.

25. The drug delivery device according to claim 24, further comprising a counter fastening element configured to mechanically engage with a complementary shaped fastening element of the electronic module in the predefined fastening configuration.

26. The drug delivery device according to claim 25, wherein the mechanical counter coding is defined by at least one of a position, an orientation, and a longitudinal extent of the mechanical counter coding feature relative to the counter fastening element.

27. The drug delivery device according to claim 24, wherein the mechanical counter coding feature comprises the recess configured to receive or to engage with a complementary shaped and distally extending protrusion of the coding feature of the mechanical coding when in the predefined fastening configuration.

28. The drug delivery device according to claim 27, wherein the recess is provided in at least one of a proximal facing surface of the drug delivery device, an outside surface of a sidewall of the drug delivery device and an inside surface of the sidewall of the drug delivery device.

29. The drug delivery device according to claim 28, further comprising a drug container filled with the drug.

30. A modular system comprising:
an electronic module configured for attachment to a proximal end of a drug delivery device in a predefined fastening configuration, the drug delivery device comprising an elongated housing extending in a longitudinal direction and comprising a distal end and the proximal end, the electronic module comprising:
a mechanical coding comprising a mechanical coding feature to engage with a mechanical counter coding feature of a mechanical counter coding provided at the proximal end of the drug delivery device,
wherein one of the mechanical coding feature and the mechanical counter coding feature comprises a protrusion extending in the longitudinal direction, and wherein the other one of the mechanical coding feature and the mechanical counter coding feature comprises a recess, and
wherein when a geometric shape of the protrusion is not matching with a geometric shape of the recess, or when a position of the protrusion in a plane transverse to the longitudinal direction is not matching with a position of the recess in the transverse plane, or when a longitudinal extent of the protrusion is larger than a longitudinal extent of the recess, the mechanical coding and the mechanical counter coding being operable to prevent a fastening of the electronic module to the proximal end of the drug delivery device in a predefined fastening configuration, and
the drug delivery device;
wherein the mechanical coding of the electronic module matches with the mechanical counter coding of the drug delivery device, and
wherein the mechanical coding mechanically engages with the mechanical counter coding when the electronic module and the drug delivery device are in the predefined fastening configuration.

31. The modular system of claim 30, wherein the electronic module further comprises a fastening element configured to mechanically engage with a complementary shaped counter fastening element of the drug delivery device in the predefined fastening configuration.

32. The modular system of claim 31, wherein the mechanical coding is defined by at least one of a position, an orientation and a longitudinal extent of the mechanical coding feature relative to the fastening element.

33. The modular system of claim 30, wherein the electronic module further comprises at least one longitudinal extension extending distally from a distal end of the electronic module and configured to extend into or through an aperture at the proximal end of the drug delivery device.

34. The modular system of claim 33, wherein the mechanical coding is defined by at least one of a position, an orientation and a longitudinal extent of the mechanical coding feature relative to the longitudinal extension.

35. The modular system of claim 30, wherein the mechanical coding feature comprises the protrusion protruding in a longitudinal distal direction from a distally facing surface of the electronic module and configured to engage with a complementary shaped recess of the mechanical counter coding feature.
